(19) United States
(12) Patent Application Publication
(10) Pub. No.: US 2021/0093556 A1
(43) Pub. Date: Apr. 1, 2021

(54) GLUTATHIONE DISULFIDE IN PARENTERAL NUTRITION FOR MAINTAINING OR INCREASING PROTEIN SYNTHESIS

(71) Applicant: VALORISATION HSJ, LIMITED PARTNERSHIP, Montreal (CA)

(72) Inventors: Jean-Claude LAVOIE, Laval (CA); Ibrahim MOHAMED, Pierrefonds (CA)

(21) Appl. No.: 16/772,215

(22) PCT Filed: Dec. 12, 2018
(86) PCT No.: PCT/CA2018/051581

§ 371 Date: Jun. 12, 2020

Related U.S. Application Data
(60) Provisional application No. 62/597,653, filed on Dec. 12, 2017.

(51) Int. Cl. A61K 9/00; A61K 38/06; A61P 3/02; A61K 9/107; A23L 33/00; A23L 33/16; A23L 33/175; A23L 33/125; A23L 33/15; A23L 33/115; A23L 33/18

(52) U.S. Cl. CPC A61K 9/0029; A61K 38/063; A61P 3/02; A61K 9/107; A23L 33/40; A23V 2002/00; A23L 33/175; A23L 33/125; A23L 33/15; A23L 33/115; A23L 33/18; A23L 33/16

(57) ABSTRACT

There is a need to increase protein synthesis in subjects receiving parenteral nutrition. The present disclosure provides oxidized glutathione (GSSG) or a GSSG analog as a supplement for parenteral nutrition to maintain, promote or increase protein synthesis in a subject receiving parenteral nutrition. The present disclosure also provides a parenteral nutrition solution as well as therapeutic uses of the parenteral nutrition supplement or the parenteral nutrition solution.

### CROSS-REFERENCE TO RELATED APPLICATIONS

The present application claims priority from U.S. provisional patent application 62/597,653 filed on Dec. 12, 2017 and incorporated herewith in its entirety.

### TECHNOLOGICAL FIELD

The present disclosure concerns supplements for parenteral nutrition for maintaining or increasing protein synthesis in subjects receiving parenteral nutrition.

### BACKGROUND

If complete nutrition cannot be ensured by the oral/enteral route, a complete or additional parenteral nutritional (PN) is indicated. In PN, the nutriments are administered as an infusion by the intravenous route. PN is necessary or advantageous in premature infants, in patients suffering from a partial or complete obstruction of the gastrointestinal tract, in patients suffering from a gastro-intestinal disorders (such as, for example, patients suffering from ulcerative colitis, Crohn's disease or severe diarrhea), in patient in the perioperative area, in intensive care patients as well in cases of sepsis. However, while being life-saving in many instances, PN is plagued with various side-effects associated with oxidative damages causing, amongst other things, a slower neural development and a reduction in alveolar function in the lungs.

PN has also been associated with a reduction in methionine adenosyltransferase (MAT) activity in the liver (Elremaly et al., 2016). The enzyme MAT is, amongst other things, responsible for the synthesis of cysteine from methionine. Thus, in subjects on PN, the transformation of methionine in cysteine is impaired. As such, by limiting the activity of MAT, PN is believed to reduce protein synthesis (at least in the liver) in subjects receiving PN. Subjects receiving PN need to sustain or increase protein synthesis either to sustain their growth or recover from disease.

A previous analysis reported that the addition of cysteine or N-acetylcysteine in PN failed to improve glutathione in preterm and reduce the incidence of bronchopulmonary dysplasia (BPD). It is also known that the cellular uptake of cysteine is immature in preterm newborns.

There is thus a need to provide a supplement that is suitable for parenteral administration that could sustain or increase protein synthesis in subjects receiving PN.

### BRIEF SUMMARY

The present disclosure concerns the use of disulphide glutathione (GSSG) or its analogs as a supplement for parenteral nutrition for increasing protein synthesis in subjects receiving parenteral nutrition.

According to a first aspect, the present disclosure provides a parental nutrition supplement for increasing protein synthesis in a subject receiving parenteral nutrition, the parental nutrition supplement comprising glutathione disulphide (GSSG) and/or a GSSG analog. In an embodiment, the parenteral nutrition supplement consists essentially of GSSG.

According to a second aspect, the present disclosure provides a parenteral nutrition solution for increasing protein synthesis in a subject, the parenteral nutrition solution comprising the parenteral nutrition supplement described herein and at least one serum electrolyte (which can be an inorganic salt such as, for example, NaCl). In an embodiment, the parenteral nutrition solution further comprises a mixture of amino acids. In yet another embodiment, the parenteral nutrition solution further comprises a carbohydrate (such as, for example dextrose). In still another embodiment, the parenteral nutrition solution further comprises a mixture of vitamins and/or a mixture of minerals. In yet another embodiment, the parenteral nutrition solution further comprises a lipid (which can be provided as a lipid emulsion).

According to a third aspect, the present disclosure provides method of increasing protein synthesis in a subject in need thereof and receiving parenteral nutrition, the method comprising administering by a parenteral route the parenteral nutrition supplement defined herein or the parenteral nutrition solution defined herein to the subject so as to increase protein synthesis in the subject. In an embodiment, the subject is on total parenteral nutrition. In yet another embodiment, the subject is a human. In still another embodiment, the subject is a pediatric subject (such as, for example a premature infant). In yet another embodiment, the subject is an adult subject.

According to a fourth aspect, the present disclosure provides the use of the parenteral nutrition supplement defined in claim defined or the parenteral nutrition solution defined herein for increasing protein synthesis in a subject. In an embodiment, the subject is on total parenteral nutrition. In yet another embodiment, the subject is a human. In still another embodiment, the subject is a pediatric subject (such as, for example a premature infant). In yet another embodiment, the subject is an adult subject.

According to a fifth aspect, the present disclosure provides the use of the parenteral nutrition supplement defined herein for the preparation of the parenteral nutrition solution defined in any herein for increasing protein synthesis in a subject. In an embodiment, the subject is on total parenteral nutrition. In yet another embodiment, the subject is a human. In still another embodiment, the subject is a pediatric subject (such as, for example a premature infant). In yet another embodiment, the subject is an adult subject.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 shows the plasma concentration of glutathione in function of the concentration of GSSG present in parenteral nutrition. Results are shown as total glutathione (GSH+GSSG; expressed as equivalent GSH) in plasma (μM) in function of the concentration of GSSG (μM) in the parenteral nutrition.

FIG. 2 shows the weight percentage of the liver when compared to the total body weight for animals having received various concentrations of GSSG in parenteral nutrition. Results are shown as the weight percentage of the liver in function of the concentration of GSSG (μM) in the parenteral nutrition.

FIG. 3 shows that the protein content in the liver in function of the concentration of GSSG present in parenteral nutrition. Results are shown as protein concentration in the liver (measured as mg / g of liver) in function of the concentration of GSSG (μM) in the parenteral nutrition.

FIG. 4 shows the correlation between the protein content in the liver and the plasmatic concentration of glutathione. Results are shown as protein concentration in the liver (measured as mg / g of liver) in function of total glutathione (GSH+GSSG; expressed as equivalent GSH) in plasma (μM).

### DETAILED DESCRIPTION

The present disclosure concerns the use of oxidized disulphide glutathione (GSSG) or its analogs for increasing cysteine availability and ultimately protein synthesis in a subject receiving parenteral nutrition. As shown in the Example below, protein content in the liver was determined in an animal model of PN as a proxy for determining the effects of various PN infusions on protein synthesis. In the Example, it was clearly shown that the administration of PN supplemented with GSSG increased the liver protein content and that as such GSSG does promote protein synthesis in subjects receiving PN. The present disclosure thus provide that in a pediatric population, GSSG or its analogs can be used to favor growth and development of the treated subjects receiving parenteral nutrition. In an adult population, GSSG or its analogs can be used to maintain or increase the lean body mass of the treated subjects receiving parenteral nutrition.

Parenteral Nutrition Supplement

The present disclosure provides a parenteral nutrition supplement. As used herein, the term “parenteral nutrition supplement” refer to an additive formulated to be included in a parenteral nutrition solution or administered during parenteral nutrition. The parenteral nutrition supplement can be provided in the form of a solid (such as, for example, a powder for reconstitution) or of a liquid (such as, for example, an aqueous solution) to be included in the parenteral nutrition solution. When the parenteral nutrition supplement is not included in the parenteral nutrition solution, it can be formulated for subcutaneous, intraareterial, intramuscular or intravenous administration. The parenteral nutrition supplement can formulated as a single dose for example a vial, with or without added preservatives or in prefilled syringes. The parenteral nutrition supplement may be in such form as suspensions, solutions or emulsions in oily or aqueous carriers and they may contain agents formulated as suspension, stabilisers and/or dispersants.

The parenteral nutrition supplement comprises GSSG or an analog of GSSG. As it is known in the art, the compound GSSG is a disulfide compounds derived from two gluthatione (GSH). GSSG (also known as oxidized glutathione) is thus a tripeptide dimer (γ-glutamyl-cysteinyl-glycine), where two molecules of the tripeptide with the aforementioned formula are linked via a covalent bond between cysteine residues.

Analogs of GSSG are structurally-related but different compounds that GSSG which are able to mediate one or more of the biological activity associated to GSSG. Analogs of GSSG are substrates for the γ-glutamyltranspeptidase enzyme and favor the intracellular release of cysteine. In the context of the present disclosure, the GSSG analogs are capable of maintaining the protein content or increasing the protein synthesis in a subject receiving parenteral nutrition. GSSG analogs includes, but are not limited to S-derivatives of glutathione, GSSG molecule chemically modified by binding covalently another compound (e.g., cysteamine-(2-mercaptoethylamine), lipoic acid (6,8-10 thioctic acid), carnosine (b-alanyl-hystidine) and adenosine (9-β-D-ribofaranosyladenine). GSSG analogs have been described in U.S. Pat. No. 6,312,734, U.S. Pat. No. 6,251,857, U.S. Pat. No. 7,371,411 and US Patent Application published under 2011/0064828. GSSG analogs can be a mixture of various structurally-related compounds or a single compound.

Upon storage, GSSG and its analogs can be formulated so as to prevent their reduction into glutathione (GSH). Stabilized formulations of GSSG are well known and some of them have been described in WO 97/21444 and U.S. Pat. No. 6,312,734. The present disclosure thus also provides pharmaceutically acceptable salts of GSSG and its analogs in the form of a parental supplement. As used in the present disclosure, the term “pharmaceutically acceptable salt” refers to conventional acid-addition salts or base-addition salts that retain the biological effectiveness and properties of GSSG or its analog and are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as e.g., tetramethylammonium hydroxide. The chemical modification of a pharmaceutical compound into a salt is a well-known technique which is used in attempting to improve properties involving physical or chemical stability, e.g., hygroscopicity, flowability or solubility of compounds. Exemplary oxidized glutathione salts were described in WO 2013/002317. For example, GSSG or its analog can be provides as a sodium salt.

Parenteral Nutrition Solution

The present disclosure also provides a parenteral nutrition solution for increasing protein synthesis in a subject. As used in the context of the present disclosure, the parenteral nutrition solution is for the intravenous feeding of a subject. The parenteral nutrition solution can be used for the total parenteral nutrition of the subject, e.g., the subject is being fed exclusivity with the parenteral nutrition solution. Alternatively, the parenteral nutrition solution can also be used for a partial parenteral nutrition of the subject (also referred as supplemental nutrition), e.g., the subject is being fed both orally and parenterally.

In the context of the present disclosure, the parenteral nutrition solution is formulated either to include the parenteral nutrition supplement described herein, to be combined with the parenteral nutrition supplement described herein or to be administered with the parenteral nutrition supplement. In an embodiment, the concentration of GSSG or its analog in the parenteral nutrition solution or co-administered with the parenteral nutrition solution is at least 4 μM, at least 5 μM, at least 6 μM or at least 7 μM. In still another embodiment, the concentration of GSSG or its analog in the parenteral nutrition solution or co-administered with the parenteral nutrition solution can be at most 8 μM, at most 7 μM, at most 6 μM or at most 5 μM. In yet another embodiment, the concentration of GSSG or its analog in the parenteral nutrition solution or co-administered with the parenteral nutrition solution is between about 4 μM, 5 μM, 6 μM or 7 μM and about 8 μM, 7 μM, 6 μM or 5 μM. In yet another embodiment, the concentration of GSSG or its analog in the parenteral nutrition solution or co-administered with the parenteral nutrition solution is between about 4 μM and about 8 μM. In still another embodiment, the concentration of GSSG or its analog in the parenteral nutrition solution or co-administered with the parenteral nutrition solution is about 4 μM.

As it is known in the art, the parenteral solution is an emulsion infused in a subject for providing parenteral nutrition. The parenteral solution is aseptically prepared and sterile upon administration to the subject. The person skilled in the art knows that the components and dosages for each components of the parenteral nutrition solution can vary between subjects or during parenteral nutrition of the same subject. The composition of the parenteral nutrition will depend on the disease status as well as the subject receiving parenteral nutrition and that certain components can be added or removed from the parenteral nutrition solution in order to accommodate the subject. The nutrients' mix of the parenteral nutrition is tailored to the subject's needs and tolerance.

The parenteral solution usually comprises serum electrolytes (such as, for example, potassium, sodium, magnesium, calcium, phosphate or a mixture thereof) in an aqueous solution.

Optionally, the parenteral solution can comprise one or more (e.g., a mixture) of amino acids for supporting protein synthesis. The amino acids present in the parental solution can include essential amino acids (such as, for example, phenylalanine, valine, threonine, tryptophan, methionine, leucine, isoleucine, lysine and histidine), conditionally essential amino acids (e.g., arginine, cysteine, glycine, glutamine, proline, and tyrosine) as well as non-essential or dispensable amino acids (e.g., alanine, aspartic acid, asparagine, glutamic acid and serine). In embodiments, the parenteral solution can also include branched side chains amino acids (e.g., leucine, isoleucine and valine). In some embodiments, modified amino acids (such as for example, ornithine) can also be included in the parenteral solution.

The parenteral solution can also include one or more carbohydrates for providing an energy source to the subject receiving parenteral nutrition. Carbohydrates which can be included in the parenteral solution include monosaccharides (such as glucose or dextrose) and/or disaccharides (such as, for example, sucrose, lactose, maltose, trehalose or a mixture thereof). In an embodiment, the parenteral solution comprises dextrose. In still another embodiment, the parenteral solution comprises dextrose as the sole carbohydrate.

The parenteral nutrition solution can also include a mixture of vitamins and/or mineral. The mixture of vitamins can include, vitamin B1 (thiamine), vitamin B2 (riboflavin), vitamin B6 (pyridoxine), vitamin B12 (cobalamin), pantothenic acid, niacin, biotin, folic acid, vitamin C, vitamin A, vitamin D, vitamin E, vitamin K or a mixture thereof. The mixture of minerals can include, but is not limited to, chromium, copper, iodine, iron, manganese, selenium, molybdenum and/or zinc.

The parenteral nutrition solution can include lipids for allowing a high energy supply, facilitating the prevention of high dextrose infusion rates and supplying essential fatty acids. In some embodiments, the lipids are provided in the form of a lipid emulsion. In some embodiment, the lipid emulsion comprises a low phospholipid/triglyceride ratio to prevent the depletion of essential fatty acids, lower the risk of hyperglycaemia and prevent hepatic steatosis. To prevent lipid peroxidation, vitamin E can be included in a parenteral nutrition solution comprising lipids.

The parenteral nutrition solution can also include active therapeutic agents or drugs. For example, when subjects receiving parenteral nutrition are at risk of hyperglycaemia (for example subjects afflicted by diabetes), the parenteral nutrition solution can comprise or be administered with insulin or an insulin derivative. In yet another example, when subjects receiving parenteral nutrition are at risk of forming a blood clot (or clots), the parenteral nutrition solution can include heparin or an heparin analog.

The present disclosure also provides a commercial kit comprising a parenteral nutrition solution as described herein and the parenteral nutrition supplement described herein. Optionally, the commercial kit also includes instructions on how to combine or administer the parenteral solution with the parenteral nutrition supplement.

Therapeutic Use of the Parenteral Nutrition Supplement and the Parenteral Nutrition Solution

The parenteral nutrition supplement described herein can be used to increase protein synthesis in a subject receiving parenteral nutrition. Subjects receiving parenteral nutrition are in need of at least maintaining their level of protein synthesis similar to the one prior to receiving parenteral nutrition. In some embodiments, subjects receiving parenteral nutrition are in need of increasing their level of protein synthesis in order to maintain or gain body mass, such as, for example, lean body mass. This maintenance or increase in protein synthesis can be seen in one or more organ (such as the liver, the lungs, the brain) or one or more tissue (such as muscles, skin, etc.). As such, the parenteral nutrition supplement described herein can be used to increase the mass (and in an embodiment, the lean mass) of an organ, a tissue or a subject.

Parenteral nutrition is used when subjects cannot be fed exclusively orally. Examples of conditions requiring parenteral nutrition (either total or supplemental) include malnutrition (from such acute and chronic inflammatory bowel diseases as regional ileitis (Crohn's disease and ulcerative colitis), partial or total obstruction of the gastrointestinal tract, prematurity, congenital anomalies in the newborn (prior to surgery), massive burns that produce critical protein loss, intestinal failure, short bowel syndrome, etc. The parenteral nutrition supplement can be used in all subjects which cannot be exclusively fed orally to maintain or increase cysteine biosynthesis and, ultimately, protein synthesis.

In the context of the present disclosure, the subject receiving parenteral nutrition can be a mammal and in some embodiments, a human. In some embodiments, the subject can be a pediatric subject or an adult subject.

Subjects are considered pediatric between their birth date up to 18 or 21 years old. Pediatric subjects include, but are not limited to, infants (such as, for example, newborns as well as premature infants), children as well as adolescents. The present disclosure is especially useful in premature infants that cannot feed entirely orally. In such patient population, the parenteral nutrition supplement described herein or the parenteral nutrition solution described herein can be used to maintain or increase the infant's weight, lean body mass and/or protein synthesis.

The present disclosure is also useful for maintaining or increasing protein synthesis in children and adolescents. In such patient population, the parenteral nutrition supplement described herein can be used maintain the growth (e.g., weight, height, body mass, including lean body mass) and development of children and adolescents who need to received parenteral nutrition. Children and adolescents are still growing and receiving parenteral nutrition may slow down their growth. As such, receiving the parenteral nutrition supplement described herein may help children and adolescents maintain their normal growth. In some instances, the parenteral nutrition supplement described herein can even be used to increase the growth and development of children and adolescents receiving parenteral nutrition.

In the methods and therapeutic uses described herein, GSSG or its analog can be provided in the parenteral nutrition solution or co-administered with the parenteral nutrition solution to provide a total glutathione plasma level of at least 4 μM or least 5 μM. In still another embodiment, GSSG or its analog can be provided in the parenteral nutrition solution or co-administered with the parenteral nutrition solution to provide a total glutathione plasma level of at most 6 μM or at most 5 μM. In yet another embodiment, GSSG or its analog can be provided in the parenteral nutrition solution or co-administered with the parenteral nutrition solution to provide a total glutathione plasma level between about 4 μM or 5 μM and about 6 μM or 5 μM. In yet another embodiment, GSSG or its analog can be provided in the parenteral nutrition solution or co-administered with the parenteral nutrition solution to provide a total glutathione plasma level between about 4 μM and about 6 μM. In still another embodiment, the can be provided in the parenteral nutrition solution or co-administered with the parenteral nutrition solution to provide a total glutathione plasma level of about 4 μM.

The present invention will be more readily understood by referring to the following examples which are given to illustrate the invention rather than to limit its scope.

### EXAMPLE

Eight groups of 6 animals were compared. At three days of age, a catheter was fixed in jugular vein of guinea pigs (Hartley, Charles River, Saint-Constant, QC, Canada) in order to receive infusion of parenteral nutrition (PN) (87 g/L dextrose, 20 g/L amino acids (Primene, Baxter, Mississauga, ON, Canada), 1% (v,v) multivitamin preparation (Multi-12 pediatrics, Sandoz, Boucherville, QC, Canada), 16 g/L final volume of Intralipid (Frenisius Kabi Canada Ltd., Richmond Hill, ON, Canada), electrolytes and 1 U/mL heparin. The PN solutions were enriched with 0 to 12 μM GSSG (Sigma-Aldrich Canada or Sandoz, Boucherville, QC, Canada). PN, continuously infused at 20 mL/100 g/d, were changed daily (Elremaly et al., 2012; Elremaly et al., 2015; Elremaly et al., 2016). A further group of animals of same age but without manipulation has served as control. After 4 days (at one week of age), animals were sacrificed to blood and liver collection. Samples were prepared according to target determinations and stored at −80° C. The approval of the Institutional Committee for the Good Practice with Animal in Research, according to the guideline of the Canadian Council on Animal Care was obtained for all these procedures.

Total glutathione in plasma was measured as followed. Immediately following its draw, blood was centrifuged at 5 000 g during 4 minutes to isolate plasma. Total glutathione (GSH +GSSG) was measured according to the method of Griffith et al. (1980) and it is presented as GSH equivalent.

Total protein content in liver was determined by using the commercial Bradford Protein Assay (Bio-rad, Hercules, Calif., USA) as follow. Immediately after its sampling, liver was homogenized (1:5; g:vol of 5% (w/v of water) metaphosphoric acid) during 20 sec on iced water with a Polytron (Biospec Products, Bartlesville, Okla., USA). After centrifugation at 5 000 g during 3 min, the pellet was frozen at −80° C. until protein determination. Thawed pellet has been diluted (1:4) in NaOH 1N in order to solubilize proteins. 24 hr later, the solution was diluted 1:250 in water before determination. BSA was used for the standard curve

Data are presented as mean±sem. ANOVA was used for orthogonal comparisons between groups. The homoscedasticity was confirmed using Bartlett's Chi². Pearson correlation was used to quantify the linear relation between plasma glutathione and hepatic protein content. The level of significance was set at p<0.05.

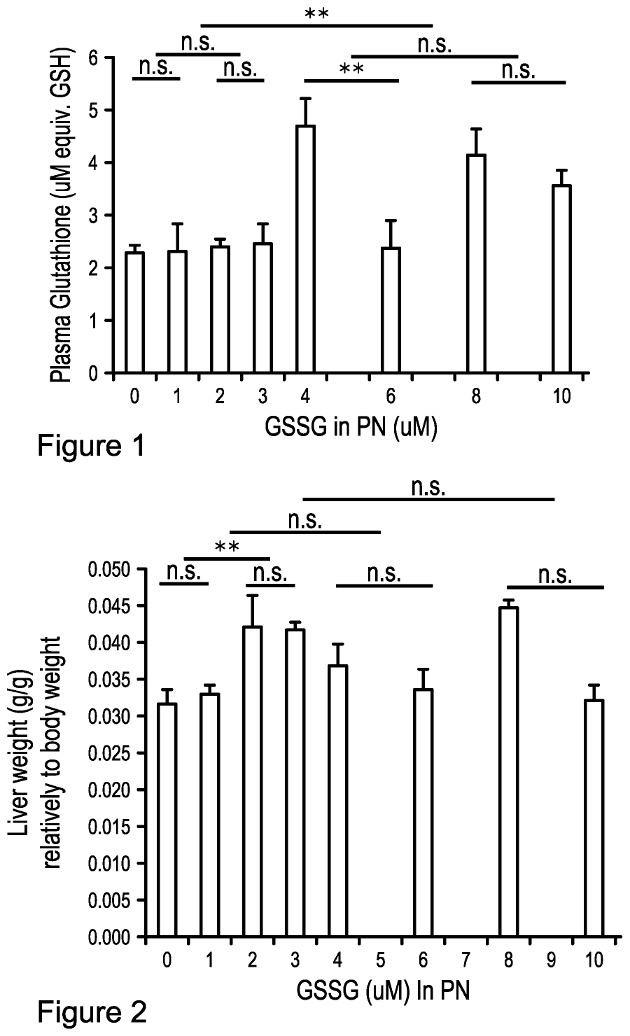

As shown on FIG. 1, no modification in plasma glutathione was observed when GSSG was provided at a concentration between 0 and 3 uM in PN. The plasma concentrations of glutathione in plasma of animals receiving ≥4 uM GSSG in PN were variable. Independently of this variation, overall, plasma concentration of glutathione was significantly greater in animals receiving PN enriched with 4 uM GSSG relatively to those with 3 uM GSSG or less.

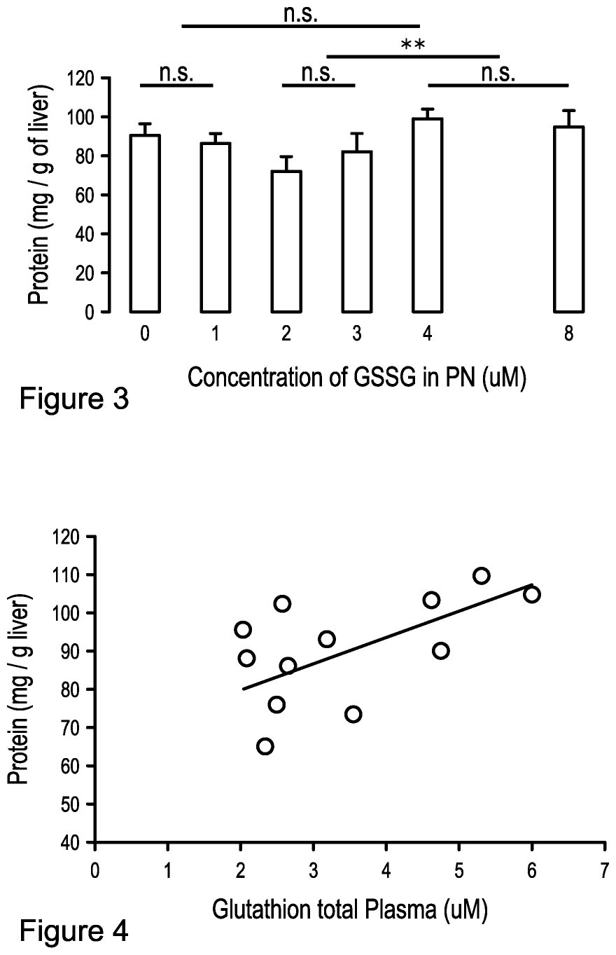

As shown in FIG. 2, the liver weight was higher in animals receiving 2 uM GSSG in PN. This weight increase was attributed to increase protein synthesis since the protein content of the liver of animals treated with GSSG remained similar to the protein content of the liver of animals that did not receive GSSG (see FIG. 3).

FIG. 4 shows that the variation in hepatic protein content is positively influenced by the concentration of total glutathione in plasma (y=6.9×mg g-1·uM-1+66.0 mg·g-1; r2=0.39, p<0.05).

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Elremaly W, Rouleau T, Lavoie JC. Inhibition of hepatic methionine adenosyltransferase by peroxides contaminating parenteral nutrition leads to a lower level of glutathione in newborn guinea pigs. Free Radic Biol Med 53:2250-2255, 2012

Elremaly W, Mohamed I, Rouleau T, Lavoie J C. Adding glutathione to parenteral nutrition prevents alveolar loss in newborn Guinea pig. Free Radic Biol Med 87:274-281, 2015

Elremaly W, Mohamed I, Rouleau T, Lavoie J C. Impact of glutathione supplementation of parenteral nutrition on hepatic methionine adenosyltransferase activity. Redox Biol. 2016 August; 8:18-23.

Griffith O W. Determination of glutathione and glutathione disulfide using glutathione reductase and 2-vinylpyridine. Anal Biochem 1980; 106:207-212.

1. A parental nutrition supplement for increasing protein synthesis in a subject receiving parenteral nutrition, the parental nutrition supplement comprising glutathione disulphide (GSSG) and/or a GSSG analog.

2. The parenteral nutrition supplement of claim 1 consisting essentially of GSSG.

3. A parenteral nutrition solution for increasing protein synthesis in a subject, the parenteral nutrition solution comprising the parenteral nutrition supplement of claim 1 or 2 and at least one serum electrolyte.

4. The parenteral nutrition solution of claim 3, further comprising a mixture of amino acids.

5. The parenteral nutrition solution of claim 3, further comprising a carbohydrate.

6. The parenteral nutrition solution of claim 5, wherein the carbohydrate is dextrose.

7. The parenteral nutrition solution of claim 3, further comprising a mixture of vitamins.

8. The parenteral nutrition solution of claim 3, further comprising a mixture of minerals.

9. The parenteral nutrition solution of claim 3, further comprising a lipid.

10. The parenteral nutrition solution of claim 9, wherein the lipid is provided as a lipid emulsion.

11. The parenteral nutrition solution of claim 3, wherein the serum electrolyte is provided as an inorganic salt.

12. The parenteral nutrition solution of claim 11, wherein the organic salt is NaCl.

13. A method of increasing protein synthesis in a subject in need thereof and receiving parenteral nutrition, the method comprising administering by a parenteral route the parenteral nutrition supplement defined in claim 1 to the subject so as to increase protein synthesis in the subject.

14. (canceled)

15. (canceled)

16. The method of claim 13, wherein the subject is on total parenteral nutrition.

17. The method of claim 13, wherein the subject is a human.

18. The method of claim 13, wherein the subject is a pediatric subject.

19. The method of claim 18, wherein the pediatric subject is a premature infant.

20. The method of claim 13, wherein the subject is an adult subject.
